(19) United States
(12) Patent Application Publication
(10) Pub. No.: US 2004/0071587 A1
(43) Pub. Date: Apr. 15, 2004

(54) Quick setup decontamination stall

(76) Inventor: Patrick F. McAtarian, St. Mary's, KS (US)

Correspondence Address: HOVEY WILLIAMS LLP, Suite 400, 2405 Grand Boulevard, Kansas City, MO, 64108, US

(21) Appl. No.: 10/269,547

(22) Filed: Oct. 11, 2002

(51) Int. Cl. A01N 1/00; A61L 2/00

(52) U.S. Cl. 422/1; 422/28; 422/292; 422/294

(57) ABSTRACT

A decontamination unit (10) is provided including an upright collapsible frame (12) and a one-piece integrated enclosure (14). The frame (12) includes extensible legs (16) interconnected by upper scissor linkages (20); it may be manually erected in the field in a very few minutes. The enclosure (14) is suspended on and supported by the frame (12) and preferably includes a lower basin floor (30) and upright wall structure (40). One or more ingress/egress passageways (70) are provided in the wall structure (40). The unit (10) may be used individually or a plurality thereof may be positioned in juxtaposition for staged decontamination purposes.

### BACKGROUND OF THE INVENTION

1. Field of the Invention

The present invention is broadly concerned with field-erectable decontamination units which may be quickly and easily installed and used at or adjacent a contaminated site for the purpose of rapid decontamination of individuals or objects. More particularly, the invention is concerned with such decontamination units and methods of use thereof wherein the units include an upright frame which supports a decontamination enclosure, the latter preferably having a liquid-collecting basin floor as well as upright wall structure. The units may be used individually or a plurality thereof may be placed in adjacency for staged decontamination operations.

2. Description of the Prior Art

In situations of industrial workplace accidents or use of biological or chemical weapons, individuals or important objects in the vicinity may become contaminated. It is often essential that decontamination procedures be carried out on a rather immediate basis, in order to minimize the risks to health and well being of affected individuals. It has been suggested in the past to provide field decontamination devices allowing application of decontaminating substances such as sprays or powders. However, these prior assemblies are often heavy and cumbersome, and difficult to erect and move. For example, heavy basins have been provided along with separate upright screens. However, owing to the non-integrated nature of these systems, they are bulky and time-consuming to install. At the same time, these units often do not provide any degree of privacy, which is important inasmuch as contaminated individuals may be required to disrobe.

There is accordingly a need in the art for improved decontamination units which can be readily erected while at the same time being rugged, impervious to contaminants and decontaminating substances, and which can be used singly or in combination.

### SUMMARY OF THE INVENTION

The present invention overcomes the problems outlined above and provides a decontamination unit including a unitary, one-piece, upright liquid-containing enclosure comprising a basin floor and upright wall structure extending upwardly from the basin floor. The enclosure is formed of a flexible material with the inner face thereof formed of synthetic resin material, and is of sufficient height to accommodate a standing adult therein. The overall unit further includes a frame supporting said enclosure which may be exterior therein or within the confines thereof.

In preferred forms, the basin floor and upright wall structure are permanently interconnected by means of heat welding or other means to form the enclosure. Although the enclosure may be initially fabricated from individual pieces, the ultimate enclosure is essentially unitary and one-piece.

The most preferred frame has a plurality of upright, telescopic legs with scissor linkages interconnecting the legs. The linkages are designed so that the entire frame may be collapsed into a compact package, while nevertheless allowing the frame to be fully erected in a minute or so. The enclosure is preferably fabricated from synthetic resin materials, with the basin floor advantageously fabricated from reinforced synthetic resin material to enhance the durability and puncture resistance thereof, whereas the wall structure may be fabricated using somewhat thinner synthetic resin material.

### BRIEF DESCRIPTION OF THE DRAWINGS

FIG. 1 is a perspective view of a decontamination unit in accordance with the invention shown during use thereof where a decontaminating spray is applied to a person (not shown) within the unit enclosure;

FIG. 2 is an enlarged, fragmentary view depicting portions of the exterior decontamination unit frame and the strap connectors preferably employed for securing the upright enclosure to the frame;

FIG. 3 is a side view of the preferred exterior unit frame in a partially collapsed condition;

FIG. 4 is a fragmentary vertical sectional view illustrating the construction of the basin floor forming a part of the enclosure, as well as the preferred drain opening; and

FIG. 5 is a plan view of a decontamination assembly made up of plural decontamination units placed in close proximity juxtaposition, allowing staged decontamination operations.

### DETAILED DESCRIPTION OF THE PREFERRED EMBODIMENT

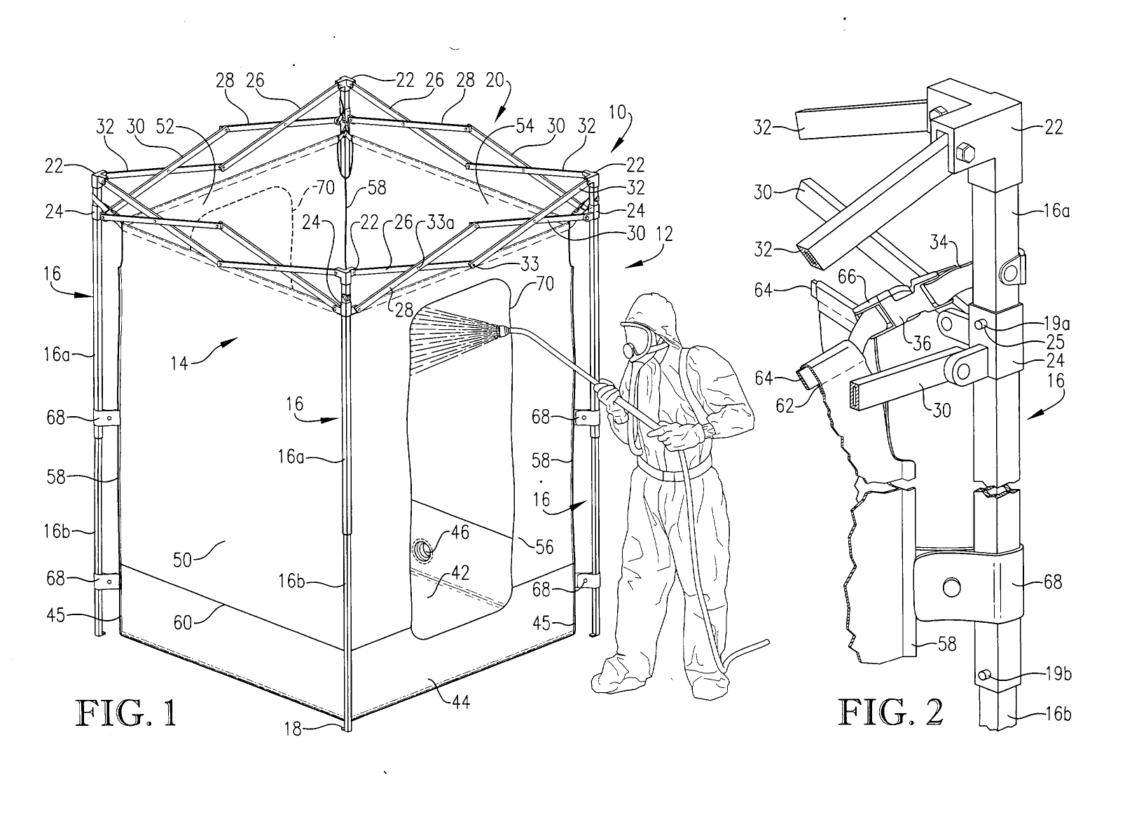

Turning now to the drawings, a decontamination unit 10 is illustrated in FIG. 1 and broadly includes an exterior frame 12 supporting an upright enclosure 14. The unit 10 is designed to be quickly field-erected and used to decontaminate individuals. As depicted in FIG. 1, a person (not shown) within enclosure 14 may be decontaminated by application of a liquid spray or other decontaminating substance, with the latter being effectively collected within the enclosure for safe disposal.

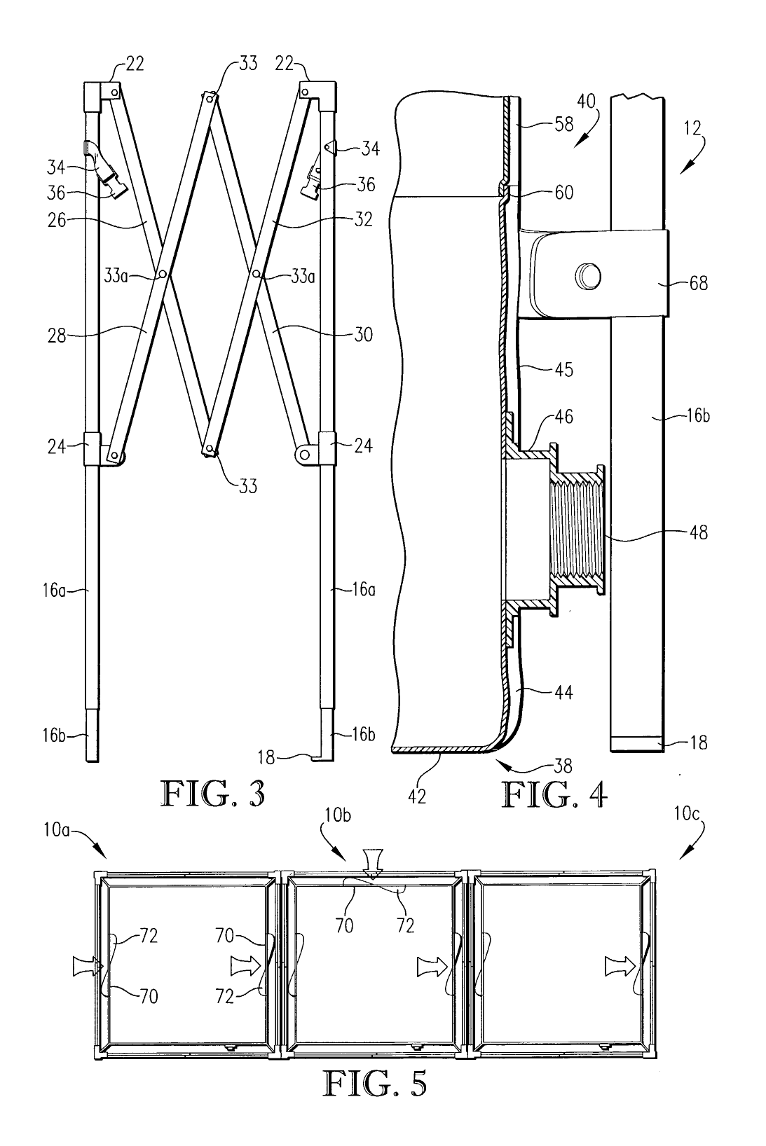

In more detail, the frame 12 in the illustrated embodiment includes a total of four upright telescopic legs 16 which are laterally spaced apart and are adapted to engage the earth or other support surface. Each leg includes an upper square tubular segment 16a as well as a telescopically interfitted lower square tubular segment 16b, the latter terminating with a short ground-engaging footpad 18. A pair of spaced apart, spring loaded locking pins 19a and 19b (FIG. 2) are provided on leg segment 16a as shown. The legs 16 are interconnected by four identical scissor linkages 20 positioned adjacent the upper ends of the legs. As illustrated in FIG. 2, each leg is equipped with an uppermost L-bracket 22 as well as a tubular, vertically reciprocal slide L-bracket 24 disposed below bracket 22 and having a locking aperture 25. Each linkage 20 is made up of four linkage arms 26-32. The legs 26 and 32 are each pivotally coupled to a bracket 22, while the arms 28 and 30 are pivotally coupled to a slide bracket 24. The arms 28 and 30 are pivotally interconnected at the ends thereof through pivot pins 33, as are the arms 26 and 32. Finally, the arms 26, 28 and 30, 32 are pivotally interconnected at their respective midpoints as shown via pivot pins 33a. As best seen in FIGS. 2 and 3, each segment 16a is provided with a connection strap 34 having one end secured beneath bracket 22 and with a female buckle connector 36 at the other end thereof. It will be appreciated from the foregoing description that the frame 12 may be moved from a collapsed, compact orientation wherein the arms of linkages 20 are generally parallel and vertically oriented to the fully extended use position shown in FIG. 1, with the slide brackets 24 accommodating the movement between the collapsed and use positions. In the extended use position the respective bracket apertures 25 receive the corresponding pins 19a, while in the collapsed position these apertures 25 receive the lower pins 19b.

The enclosure 14 broadly includes a basin floor 38 as well as upright wall structure 40. The floor 38 is preferably formed from a synthetic resin material having a thickness of from about 15-50 mil, and is most preferably fabricated from commercially available fiber cord-reinforced triple laminate extruded or blown polyethylene having a thickness of about 27 mil. As illustrated, the drain floor 38 includes a bottommost generally horizontal bottom panel 42 as well as an upright circumscribing sidewall section 44, the latter heat welded to present vertical corner joints 45. In this fashion, the floor 38 serves as a liquid collection basin. As best seen in FIG. 4, the sidewall section 44 is equipped with a hard synthetic resin drain fitting 46 provided with a threaded outer section 48. A cap (not shown) may be threaded into the section 48 during use of the unit 10, and alternately a drainage line equipped with a pump may be secured into and through fitting 46 to allow drainage of collected liquid from the drain floor 38.

The wall structure 40 is likewise preferably formed from sheet synthetic resin material, typically having a thickness of from about 5-25 mil, and most preferably a blown polyethylene film having a thickness of 10 mil. The wall structure is fabricated from four generally rectangular sheets 50-56 having vertical marginal heat weld seals 58, thereby presenting a substantially quadrate configuration in plan. The lower margin of the wall structure 40 is similarly heat welded to the upper margin of the wall section 44, thus defining a circumscribing joint 60. The upper margins of the respective sheets 50-56 are folded over and sealed, thereby defining elongated, horizontally extending passageways 62. A continuous mounting strap 64 formed of canvas or other suitable material passes through the passageways 62, and has a male buckle connector 66 at each corner thereof, which are designed to mate with the connectors 36 described above.

Each of the corner seals 58 also have two vertically spaced apart connector straps 68 of length to wrap about the adjacent portion of a leg 16; the straps 68 each have a conventional snap connector arrangement as depicted permitting connection to the leg.

In order to permit ingress and egress from the unit 10, a passageway 70 is provided in one of the wall structure sheets 50-56, it being noted that the bottom of the passageway is above bottom panel 42 to retain the liquid-holding capacity of the basin floor 38. Also, other similar passageways can be provided in other wall structure sheets, such as shown in phantom in FIG. 1, depending upon the desired use of the unit in question. In preferred embodiments, perforations may be made in portions of wall structure 40 so that additional passageways may be formed by punching out the perforated wall portions. Providing perforated wall sections enables the unit 10 to be used in a staged decontamination procedure in coordination with additional units.

The erection of the unit 10 is very easy and can be accomplished in a matter of a very few minutes. As delivered, the unit is housed within a flexible bag, containing the frame 12 in its collapsed condition, as well as the enclosure 14. The first step is to erect the frame 12, involving separation of the scissor linkages 20 by manually shifting the legs 16 until the slide brackets 24 move upwardly and the pins 19a are received within the bracket apertures 25. Next, the legs 15 are elongated by extension of the sections 16b relative to the upper section 16a. At this point, the enclosure 14 is mounted on the erected frame. This involves first snapping the corner-mounted male buckle connectors 66 into the connectors 36 secured to the corner straps 34. Next, the individual straps 68 are wrapped about the adjacent legs 16 and snapped together. This creates the enclosure 10.

During decontamination use of the enclosure 10, an individual to be decontaminated steps into the unit through passageway 70. At this point, a decontaminating substance is applied, such as through the spray equipment illustrated in FIG. 1. After such decontamination, the treated individual leaves the unit through the passageway 70.

It will be appreciated that while a single decontamination unit 10 can be employed as depicted in FIG. 1, the construction thereof allows plural units to be positioned in adjacency, for a staged decontamination procedure. This utility is illustrated in FIG. 5, wherein three individual decontamination units 10a, 10b and 10c are placed in adjacency. The unit 10a includes two passageways 70 each having a protective and openable flap 72; it will be observed that the passageways are on opposite sides of the enclosure. The central unit 10b is similar to 10a, except that includes a third passageway 70 covered by openable flap 72. The final unit 10c is identical to the unit 10a. It will be understood that the assembly illustrated in FIG. 5 can be used for decontamination purposes, but that this design affords a degree of flexibility not inherent in a single unit. That is, the FIG. 5 assembly can be used for application of successive, different decontaminating substances in each of the units. Another utility would be the use of the unit 10a as a derobing station, the unit 10b as a decontamination station, and the final unit 10c as a rerobing station. It will further be appreciated that the respective adjacent passageways 70 of the plural units are arranged in close proximity and in general registry. In this fashion, an individual may move from one decontamination unit to the next by a single stride through the adjacent passageways.

While preferred embodiments of the decontamination units are illustrated, it will be appreciated that a number of different alternatives are possible. To give but a few examples, the units may be provided with roof structure, and/or the frames may be mounted inside the enclosure rather than exteriorly thereof. Also, while a generally quadrate shape has been depicted, virtually any plan configuration shape can be used. Finally, a wide variety of other materials can be used in lieu of the preferred synthetic resin materials. Basically, so long as the inner surface of the enclosure 14 presents a surface resistant to contamination and degradation by decontamination substances, it may be used in the context of the invention.

I claim:

1. A decontamination unit comprising:
a unitary, one-piece, upright liquid-containing enclosure comprising a basin floor and upright wall structure extending upwardly from the basin floor and having a passageway therethrough,
said enclosure formed of a flexible material with the inner face thereof formed of synthetic resin material,
said enclosure of sufficient height to accommodate a standing adult therein; and
a frame supporting said enclosure.

2. The unit of claim 1, said basin floor and upright wall structure being permanently interconnected to form said enclosure.

3. The unit of claim 1, said basin floor formed of a fiber cord-reinforced synthetic resin material having a thickness of from about 15-50 mil.

4. The unit of claim 3, said material being fiber cord-reinforced extruded or blown polyethylene having a thickness of about 27 mil.

5. The unit of claim 3, said upright wall structure formed of synthetic resin film material having a thickness of from about 5-25 mil.

6. The unit of claim 5, said synthetic resin film material being blown polyethylene film having a thickness of about 10 mil.

7. The unit of claim 5, said basin floor material being heat sealed to said synthetic resin film material.

8. The unit of claim 1, said basin floor including a liquid drain opening.

9. The unit of claim 1, said enclosure having an open top.

10. The unit of claim 1, said passageway presenting a bottom margin spaced above the bottom of said basin floor.

11. The unit of claim 1, said frame disposed exteriorly of said enclosure, and comprising a plurality of upright, spaced apart, interconnected legs, there being connectors for releasably coupling said enclosure to said legs.

12. The unit of claim 11, said connectors comprising strap members secured to said enclosure.

13. The unit of claim 11, said legs interconnected by collapsible scissor linkages.

14. The unit of claim 1, said enclosure being generally quadrate in plan configuration.

15. The unit of claim 1, said wall structure presenting a pair of opposed passageways therethrough.

16. A decontamination assembly comprising:
a plurality of individual decontamination units positioned in juxtaposed, close proximity relationship, each of said units including—
a unitary, one-piece, upright liquid-containing enclosure comprising a basin floor and upright wall structure extending upwardly from the basin floor,
said enclosure formed of a flexible material with the inner face thereof formed of synthetic resin material,
said enclosure of sufficient height to accommodate a standing adult therein; and
a frame supporting said enclosure,
the adjacent wall structures of said units each having a passageway therethrough which are in general alignment, permitting a person to move from one unit to the next by a single step through the generally aligned passageways.

17. The assembly of claim 16, the basin floor and upright wall structure of each of said units being permanently interconnected to form said enclosure.

18. The assembly of claim 16, the basin floor of each of said units formed of a fiber cord-reinforced synthetic resin material having a thickness of from about 15-50 mil.

19. The assembly of claim 18, said material being fiber cord-reinforced extruded or blown polyethylene having a thickness of about 27 mil.

20. The assembly of claim 18, the upright wall structure of each of said units formed of synthetic resin film material having a thickness of from about 5-25 mil.

21. The assembly of claim 20, said synthetic resin film material being blown polyethylene film having a thickness of about 10 mil.

22. The assembly of claim 20, said basin floor material being heat sealed to said synthetic resin film material.

23. The assembly of claim 16, the basin floor of each of said units including a liquid drain opening.

24. The assembly of claim 16, the enclosures of each of said units having an open top.

25. The assembly of claim 16, the passageways of said units each presenting a bottom margin spaced above the bottom of the associated basin floor.

26. The assembly of claim 16, the frames of each of said units disposed exteriorly of the corresponding enclosures, each of said frames comprising a plurality of upright, spaced apart, interconnected legs, there being connectors for releasably coupling said corresponding enclosure to said legs.

27. The assembly of claim 26, said connectors comprising strap members secured to said corresponding enclosures.

28. The assembly of claim 26, said legs interconnected by collapsible scissor linkages.

29. The assembly of claim 16, the enclosures of each of said units being generally quadrate in plan configuration.

30. A decontamination method comprising the steps of:
providing a decontamination unit comprising—
a unitary, one-piece, upright liquid-containing enclosure comprising a basin floor and upright wall structure extending upwardly from the basin floor and having a passageway therethrough,
said enclosure formed of a flexible material with the inner face thereof formed of synthetic resin material,
said enclosure of sufficient height to accommodate a standing adult therein; and
a frame supporting said enclosure;
causing a person to be decontaminated to enter said enclosure through said passageway; and
applying a decontaminating substance onto said person within said enclosure.

31. The method of claim 30, said substance comprising a liquid, said method including the step of collecting said liquid within said basin floor during said decontamination, and thereafter draining the liquid through a drain opening provided in the basin floor.

32. The method of claim 30, including the step of spraying decontaminating liquid onto said person through said passageway.
